(19) United States
(12) Patent Application Publication
(10) Pub. No.: US 2026/0295235 A1
(43) Pub. Date: Oct. 1, 2026

(54) CIRCULATORY SUPPORT SYSTEM INCLUDING STERILE SLEEVE VIEWING WINDOW

(71) Applicant: Boston Scientific Scimed, Inc., Maple Grove, MN (US)

(72) Inventors: Judith Tiferes Wang, Shoreview, MN (US); Daniel Frank Massimini, Brooklyn Park, MN (US); Nicholas Lee Tassoni, Andover, MN (US); Juliet Davida Crosby-Leonard, Quincy, MA (US)

(73) Assignee: Boston Scientific Scimed, Inc., Maple Grove, MN (US)

(21) Appl. No.: 19/576,730

(22) Filed: Mar. 24, 2026

Related U.S. Application Data
(60) Provisional application No. 63/777,419, filed on Mar. 25, 2025.

(51) Int. Cl. A61M 60/174; A61M 60/13; A61M 60/865; A61M 60/90

(52) U.S. Cl. CPC A61M 60/174; A61M 60/13; A61M 60/865; A61M 60/90; A61M 2205/583; A61M 2210/125

(57) ABSTRACT

A mechanical circulatory support system including a vascular access sheath, a percutaneous circulatory support device positionable through the vascular access sheath, and a sterile sleeve configured to be coupled to a hub of the vascular access sheath and extending proximally therefrom over the elongate shaft to form a sterile barrier. The sterile sleeve includes a tubular sleeve having a plurality of bunched locations providing the tubular sleeve with an adjustable length and a transparent tubular member having a fixed length extending distally of the tubular sleeve. One or more of the position markings formed along a length of the elongate shaft of the percutaneous circulatory support device is visible through the transparent tubular member to denote a position of the blood pump relative to the hub of the vascular access sheath.

### CROSS-REFERENCE TO RELATED APPLICATION

This application claims the benefit of U.S. Patent Application Serial No. 63/777,419, filed March 25, 2025, entitled "CIRCULATORY SUPPORT SYSTEM INCLUDING STERILE SLEEVE VIEWING WINDOW”, which is incorporated by reference herein in its entirety.

### TECHNICAL FIELD

The present disclosure pertains to a sheath configuration for a percutaneous circulatory support system. More particularly, the present disclosure pertains to a sterile sleeve of a percutaneous circulatory support system including a viewing window for confirming the position of the blood pump of the circulatory support system at a cardiovascular location during use.

### BACKGROUND

In various medical procedures a medical device, such as a percutaneous circulatory support device is advanced through a vascular access sheath to a desired position within a patient’s body. The medical device may be maintained in the patient’s body for an extended period of time. It is desirable to confirm the correct position of the medical device (e.g., the blood pump of the percutaneous circulatory support device) over a period of time while the medical device is being used to ensure that the medical device has not been inadvertently moved which may impact its efficiency and effectiveness. It may also be desirable to visually determine the position of the medical device such that controlled/gauged position adjustments of the medical device may be achieved. Accordingly, there remains an ongoing need to provide alternative systems and methods for visually determining the position of the medical device relative to the vascular access sheath to confirm proper positioning of the medical device throughout its use.

### BRIEF SUMMARY

This disclosure provides design, material, manufacturing method, and use alternatives for medical devices, including percutaneous blood pumps and associated devices.

A first example may include a percutaneous circulatory support system comprising a blood pump configured to pump blood from a ventricle of a heart of a patient to vasculature of the patient, a housing, an elongate shaft coupled with the blood pump and extending proximally from the blood pump to the housing, and a sterile sleeve configured to extend over the elongate shaft distal of the housing. The sterile sleeve includes a tubular sleeve having an adjustable length and a transparent tubular member extending distally of the tubular sleeve. The elongate shaft is visible through the transparent tubular member.

Alternatively or additionally to any of the examples above, the transparent tubular member has a fixed length.

Alternatively or additionally to any of the examples above, the elongate shaft includes a plurality of position markings. The plurality of position markings include major markings having numeric values. At least one of the major markings having numeric values is visible through the transparent tubular member regardless of the axial position of the elongate shaft therein.

Alternatively or additionally to any of the examples above, the major markings having numeric values are spaced 5 centimeters apart, and wherein the fixed length of the transparent tubular member is 5 centimeters or more.

Alternatively or additionally to any of the examples above, the transparent tubular member includes a magnification window.

Alternatively or additionally to any of the examples above, the magnification window has a magnification power of 1.5X or more.

Alternatively or additionally to any of the examples above, the tubular sleeve includes a plurality of bunched locations.

Alternatively or additionally to any of the examples above, the transparent tubular member includes an index marking configured to be used as a reference point of an axial position of the elongate shaft within the transparent tubular member.

Alternatively or additionally to any of the examples above, the elongate shaft includes a plurality of position markings, wherein the position of one of the plurality of position markings relative to the index marking is visible through the transparent tubular member.

Alternatively or additionally to any of the examples above, the transparent tubular member includes a transparent viewing window surrounded by a non-transparent tube.

Alternatively or additionally to any of the examples above, the transparent tubular member is formed of a transparent polymeric material permitting visualization of the tubular shaft therein and the tubular sleeve is formed of a translucent material obscuring visibility of the tubular shaft therein.

Alternatively or additionally to any of the examples above, the transparent tubular member has a Haze % value of less than 30% in accordance with ASTM D-1003, and the tubular sleeve has a Haze % value of greater than the Haze % value of the transparent tubular member.

Alternatively or additionally to any of the examples above, the transparent tubular member has a Haze % value of less than 30%, and the tubular sleeve has a Haze % value of greater than 30% in accordance with ASTM D-1003.

Alternatively or additionally to any of the examples above, the percutaneous circulatory support system further includes a vascular access sheath including a hub and an elongate tubular shaft extending distally from the hub. The sterile sleeve is coupled to the hub of the vascular access sheath via a connector.

Alternatively or additionally to any of the examples above, the transparent tubular member is located at a fixed distance proximal of the hub.

Alternatively or additionally to any of the examples above, the transparent tubular member includes an index marking located at a fixed distance proximal of the hub.

Another example is a percutaneous circulatory support system. The system includes a percutaneous circulatory support device and a sterile sleeve. The percutaneous circulatory support device includes a blood pump configured to pump blood from a ventricle of a heart of a patient to vasculature of the patient, a housing, and an elongate shaft coupled with the blood pump and extending proximally from the blood pump to the housing. The elongate shaft includes a plurality of position markings formed along a length of the elongate shaft. The sterile sleeve is configured to extend over the elongate shaft distal of the housing. The sterile sleeve includes a tubular sleeve having a plurality of bunched locations providing the tubular sleeve with an adjustable length and a transparent tubular member extending distally of the tubular sleeve. The transparent tubular member has a fixed length. One or more of the position markings is visible through the transparent tubular member to denote a position of the percutaneous circulatory support device.

Alternatively or additionally to any of the examples above, the plurality of position markings including major markings having numeric values. At least one of the major markings having numeric values is visible through the transparent tubular member regardless of the axial position of the elongate shaft therein.

Alternatively or additionally to any of the examples above, the transparent tubular member includes an index marking configured to be used as a reference point of an axial position of the elongate shaft within the transparent tubular member. The position of one of the plurality of position markings relative to the index marking is visible through the transparent tubular member.

Another example is a percutaneous circulatory support system including a percutaneous circulatory support device, a vascular access sheath including a hub and an elongate tubular shaft extending distally of the hub, and a sterile sleeve. The percutaneous circulatory support device includes a blood pump configured to pump blood from a ventricle of a heart of a patient to vasculature of the patient, a housing, and an elongate shaft coupled with the blood pump and extending proximally from the blood pump to the housing. The elongate shaft includes a plurality of position markings formed along a length of the elongate shaft. The sterile sleeve is configured to extend over the elongate shaft distal of the housing and be coupled to the hub of the vascular access sheath with the elongate shaft of the percutaneous circulatory support device extending through the vascular access sheath. The sterile sleeve includes a tubular sleeve formed of a non-transparent material and a transparent tubular member extending distally of the tubular sleeve. The transparent tubular member comprises a transparent material. One or more of the position markings is visible through the transparent tubular member to denote a position of the blood pump of the percutaneous circulatory support device relative to the hub of the vascular access sheath.

Alternatively or additionally to any of the examples above, the transparent tubular member includes an index marking configured to be used as a reference point of an axial position of the elongate shaft within the transparent tubular member.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a schematic perspective view of an illustrative percutaneous circulatory support device including a percutaneous blood pump;

FIG. 2 is a schematic partial cross-section view of a percutaneous circulatory support system including the percutaneous circulatory support device of FIG. 1, a sterile sleeve, and a vascular access sheath;

FIG. 3 is a schematic plan view of a distal end region of a sterile sleeve coupled to a hub of a vascular access sheath;

FIG. 4 is a schematic plan view of a distal end region of an exemplary sterile sleeve coupled to the hub of a vascular access sheath;

FIG. 5 is a schematic plan view of a distal end region of an exemplary sterile sleeve coupled to the hub of a vascular access sheath;

FIG. 6 is a schematic plan view of a distal end region of an exemplary sterile sleeve coupled to the hub of a vascular access sheath;

FIG. 7 is a schematic plan view of a distal end region of an exemplary sterile sleeve coupled to the hub of a vascular access sheath;

FIG. 8 is a cross-sectional view taken along line 8-8 of FIG. 7;

FIG. 9 is a cross-sectional view taken along line 9-9 of FIG. 7; and

FIG. 10 is a schematic plan view of a distal end region of an exemplary sterile sleeve coupled to the hub of a vascular access sheath.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

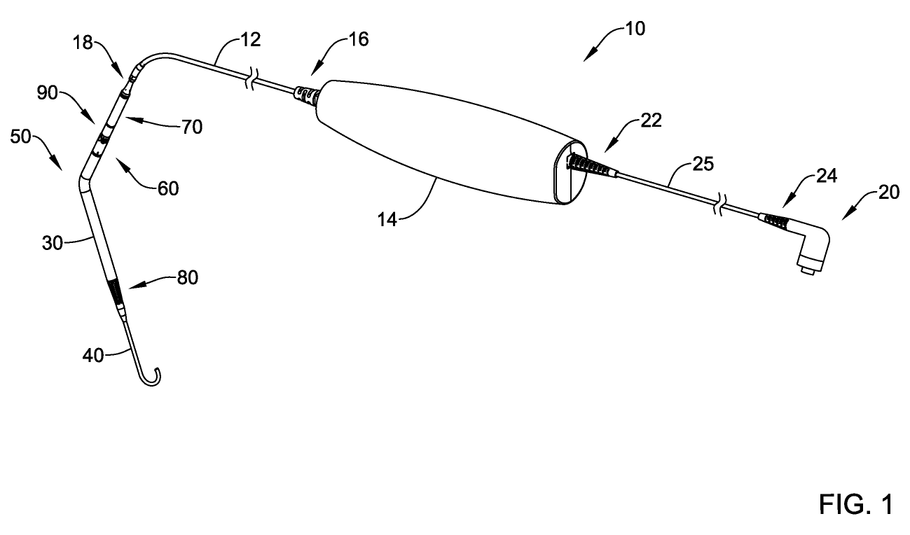

FIG. 1 illustrates a perspective view of a catheter 10 (e.g., a percutaneous circulatory support device and/or other suitable catheter) including a percutaneous blood pump 50 located at a distal end region thereof. Although not required, the blood pump 50 may be configured to pump blood from a ventricle of a heart of a patient to vasculature of the patient.

The blood pump 50 may generally include a flexible cannula 30, an impeller housing 60, and a motor housing 70. In some embodiments, the flexible cannula 30, the impeller housing 60 and/or the motor housing 70 may be integrally or monolithically constructed. In other instances, the flexible cannula 30, the impeller housing 60 and/or the motor housing 70 may be separate components. The impeller housing 60 carries an impeller assembly (not shown) therein. The impeller assembly may include an impeller secured to an impeller shaft that rotates relative to the impeller housing 60 to drive blood through the blood pump 50. In some embodiments, the impeller shaft and the impeller of the impeller assembly may be integrally formed, whereas, in other embodiments the impeller shaft and the impeller may be separate components.

Rotation of the impeller causes blood to flow from a blood inlet 80 of the blood pump 50, such as at a distal end of the flexible cannula 30, through the flexible cannula 30 and the impeller housing 60, and out of a blood outlet 90 proximal of the impeller, such as through a sidewall formed on the impeller housing 60. In some instances, the blood inlet 80 may include a plurality of blood inlet windows arranged around a circumference of the blood pump 50 (e.g., the flexible cannula 30). In some instances, the blood outlet 90 may include a plurality of blood outflow windows arranged around a circumference of the impeller housing 60. In other embodiments, the inlet 80 and/or the outlet 90 may be formed on other portions of the blood pump 50.

The percutaneous circulatory support device 10 may be coupled to or may include the blood pump 50, with an elongate shaft 12 (e.g., an elongate tube with one or more elongate members extending therein) of the percutaneous circulatory support device 10 coupled with and extending proximally from the percutaneous blood pump 50 and a distal tip 40 extending distally from the blood pump 50. For instance, a proximal end 16 of the elongate shaft 12 may be coupled to a housing 14 (e.g., a housing of a handle, of a control module, a junction box, etc.) and a distal end 18 of the elongate shaft 12 may be coupled to the percutaneous blood pump 50.

A cable 25 may extend from the housing 14 to a connector 20 at a proximal end thereof. Thus, the cable 25 may have a first, distal end 22 connected to the housing 14 and a second, proximal end 24 connected to the connector 20. The connector 20 may be configured to be connected to a controller (not shown) for controlling the blood pump 50, such as providing electrical power and/or control signals to the blood pump 50. The cable 25 may include one or more of, an electrical pathway for powering the motor within the motor housing 70, an electrical pathway for powering one or more sensors associated with the blood pump 50, an electrical pathway for transmitting signals from one or more sensors associated with the blood pump 50, a fiber optic filament for transmitting signals from one or more sensors associated with the blood pump 50, or other communication conduits. In some instances, the percutaneous circulatory support device 10 may include additional cables extending from the housing 14 configured to be connectable to the controller for sending and/or receiving signals, such as from one or more sensors during operation of the blood pump 50.

When placing the blood pump 50 in a subject (e.g., a patient), the blood pump 50 may be inserted through an introducer sheath inserted into a vascular access site of the subject. In some cases, the introducer sheath may be a large bore sheath configured to receive and facilitate the passage of the blood pump 50 and the elongate shaft 12 through a lumen thereof and into a blood vessel (e.g., a femoral artery, etc.) of the subject.

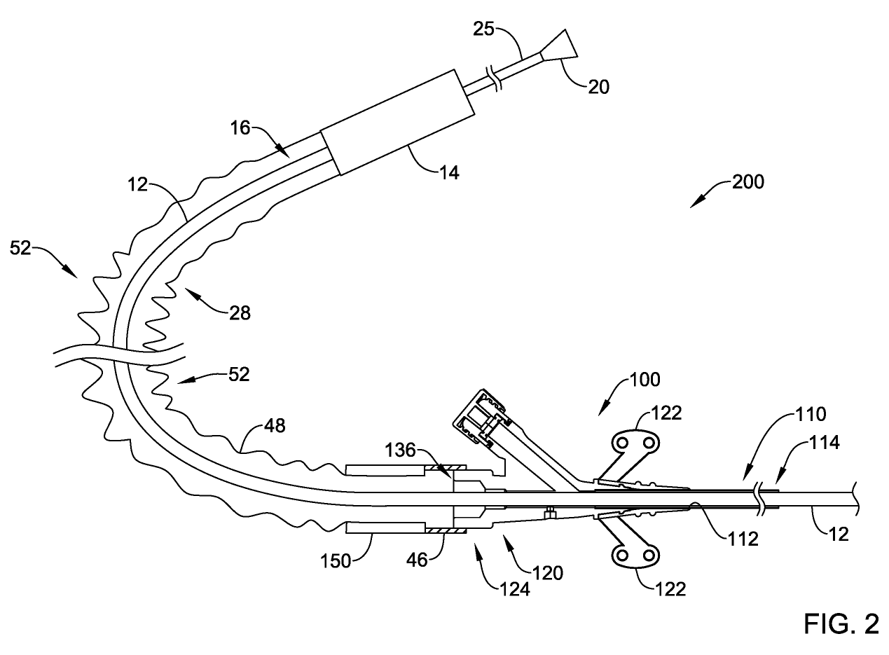

FIG. 2 depicts a schematic partial cross-section view of an illustrative configuration of a proximal end of the percutaneous circulatory support device 10 positioned within a vascular access sheath 100 (e.g., an introducer sheath or repositioning sheath). In some cases, the vascular access sheath 100 may be used for facilitating passage of various medical devices, such as the elongate shaft 12, the blood pump 50, etc., through a vascular access site of the subject and into a blood vessel of the subject. In some examples, the vascular access sheath 100 may be configured to extend into the blood vessel of the subject and allow the elongate shaft 12, the blood pump 50, and/or other suitable medical devices to pass through.

The vascular access sheath 100 may include an elongate tubular shaft 110 extending distally from a hub 120 of the vascular access sheath 100. The elongate shaft 110 may define a lumen 112 of the vascular access sheath 100 extending to a distal opening 114 at the distal end of the elongate shaft 110, through which the elongate shaft 12 of the percutaneous circulatory support device 10, the blood pump 50, and/or other suitable medical devices to pass through. Further, the vascular access sheath 100 may include a proximal opening 136 at or proximate the proximal end region of the hub 120 providing access to the lumen 112 of the elongate shaft 110.

The vascular access sheath 100 may be formed from one or more suitable materials. Example suitable materials include, but are not limited to, polymeric and/or metallic materials. In some instances, the vascular access sheath 100 may include one or more additional surface coatings on the interior surface defining the lumen 112 and/or the exterior surface, which may include, but are not limited to, silicone, polyethylene terephthalate (PET), and/or other suitable coating materials.

The hub 120 (e.g., a hub assembly such as hemostasis valve hub and/or other suitable hub assembly) of the vascular access sheath 100 (e.g., at the proximal end region thereof) may provide access to the lumen 112. The hub 120 may be configured for hemostasis by, for example, preventing or mitigating blood leakage out of the vascular access sheath 100 during use of the vascular access sheath 100. For example, a medical device, such as the elongate shaft 12 of the percutaneous circulatory support device 10 and/or the blood pump 50, may be inserted through the hub 120 and the lumen 112 of the vascular access sheath 100 and into the blood vessel of the subject, and the hub 120 may maintain hemostasis between the medical device, the vascular access sheath 100, and the external surroundings.

A sterile sleeve 28 may be provided with the percutaneous circulatory support device 10 such that the proximal portion of the elongate shaft 12 extending proximally of the vascular access sheath 100 is surrounded by the sterile sleeve 28 to maintain a sterile field. For example, a proximal end of the sterile sleeve 28 may be secured to the housing 14 and extend distally therefrom and a distal end of the sterile sleeve 28 may be securable to the hub 120 of the vascular access sheath 100. The combination of the percutaneous circulatory support device 10, the sterile sleeve 28 and the vascular access sheath 100 may be considered a percutaneous circulatory support system 200. The percutaneous circulatory support device 10 is shown in plan view in FIG. 2, while the sterile sleeve 28 and the vascular access sheath 100 are shown in cross-section. Thus, the elongate shaft 12 may extend distally from the housing 14 through the sterile sleeve 28, into the hub 120 of the vascular access sheath 100 via the proximal opening 136, and through the lumen 112 of the elongate shaft 110 of the vascular access sheath 100.

The proximal portion of the elongate shaft 12 that extends distally from the housing 14 may extend through the sterile sleeve 28. Thus, the sterile sleeve 28, which may extend distally from the housing 14, may surround the entire length of the elongate shaft 12 that is positioned proximal of the hub 120 to maintain a sterile field during about the elongate shaft 12 use of the percutaneous circulatory support system 200 during a medical procedure.

In some configurations of the sterile sleeve 28, the sterile sleeve 28 may include a distal connector 46 and a tubular sleeve 48 extending proximally from the distal connector 46 to the housing 14. The distal connector 46 may be securable (e.g., removably couplable) to the hub 120 (e.g., via a friction fit connection, a luer connection, a clip or clasp connection, threads, and/or other suitable type of connection) and create a sterile zone along the elongate shaft 12 between the proximal end of the vascular access sheath 100 and the distal end of the housing 14 of the percutaneous circulatory support device 10. The tubular sleeve 48 of the sterile sleeve 28 may be flexible, axially collapsible and/or axially elongatable to a desired length, and may be configured to have bunched locations 52 (e.g., pleats, folds, wrinkles, etc.) depending on the length of the elongate shaft 12 extending proximally from the hub 120 of the vascular access sheath 100, as depicted, for example, in FIG. 2. A bunched location 52 may be a location along the sterile sleeve 28 where excess portions of the tubular sleeve 48 may be grouped or gathered together. Thus, the bunched locations 52 may allow for the length of the sterile sleeve 28 between the housing 14 and the hub 120 of the vascular access sheath 100 to be adjusted depending on the placement of the percutaneous circulatory support device 10 during a medical procedure. Accordingly, the tubular sleeve 48 of the sterile sleeve 28 may have a variable or adjustable length.

The tubular sleeve 48 of the sterile sleeve 28, or portions thereof, may be formed from any suitable materials. Example suitable material for forming the tubular sleeve 48 include, but are not limited to, polyamide, polyurethane, thermoplastic polyurethane (TPU), polyethylene (PE), polyethylene terephthalate (PET), polypropylene (PP), ethylene vinyl acetate (EVA), polytetrafluoroethylene (PTFE), fluorinated ethylene propylene (FEP), polyvinyl chloride (PVC), polyether block amides (PEBA), and/or other suitable materials.

After advancing the percutaneous circulatory support device 10 through the vascular access sheath 100 and positioning the blood pump 50 of the percutaneous circulatory support device 10 at a target site within the subject (e.g., across the aortic valve of the heart of the patient), fixation of the axial position of the percutaneous circulatory support device 10 may be desired to ensure that the blood pump 50 is maintained in a proper position during use.

It may also be desired for medical personnel to reposition the medical device after insertion. As such, the hub 120 may include an adjustable tightening port 124, provided with or attachable thereto, that provides for the selectable fixation of the percutaneous circulatory support device 10 (e.g., the elongate shaft 12) with respect to the hub 120. The tightening port 124 of the hub 120 may be any suitable type of tightening port. For example, the tightening port 124 may tighten around and/or release from the elongate shaft 12 in response to rotating a rotational actuator of the tightening port 124, adjusting a clip of the tightening port 124, and/or the tightening port 124 may adjust about the elongate shaft 12 in one or additional or alternative manners.

To affix the vascular access sheath 100 relative to the access site of the subject, the hub 120 may include one or more suture pads 122 (e.g., two suture pads 122 extending from the hub 120, as depicted in FIG. 2) that may be sutured and/or otherwise coupled to a skin surface of the subject.

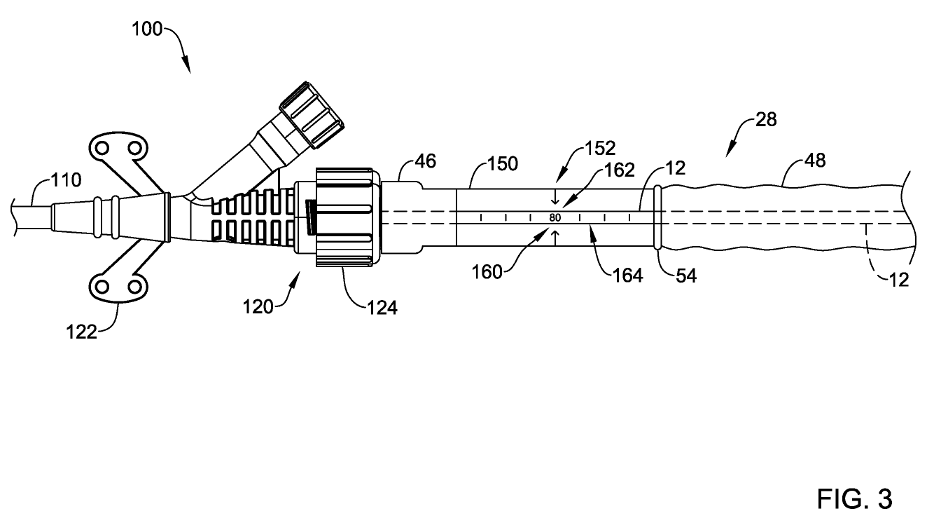

FIG. 3 depicts a distal end region of the sterile sleeve 28 coupled to the hub 120 of the vascular access sheath 100, while the elongate shaft 12 of the percutaneous circulatory support device 10 is positioned through the lumen of the vascular access sheath 100 and through the lumen of the sterile sleeve 28. As shown in FIG. 3, the distal end of the sterile sleeve 28 can include a connector 46 to removably couple the sterile sleeve 28 to the hub 120. As shown in FIG. 3, the connector 46 may include a slot into which a post of the hub 120 may extend into and engage with, to form a bayonet style connection, for example. Other connections such as, but not limited to, a threaded connection, a snap fit connection, etc. are also contemplated.

The sterile sleeve 28 may include a transparent tubular member 150 positioned along a portion of the length of the sterile sleeve 28. For example, the transparent tubular member 150 may be affixed to and extend proximal of the distal connector 46. The tubular sleeve 48 may extend proximally from the transparent tubular member 150. The transparent tubular member 150 may be distinct from the tubular sleeve 48. For example, the transparent tubular member 150 may be formed separately and connected to the tubular sleeve 48 via a connection 54 between the transparent tubular member 150 and the tubular sleeve 48. The connection 54 may be such that the transparent tubular member 150 is fixedly secured to the tubular sleeve 48, or the connection 54 may be such that the transparent tubular member 150 may be decoupled form the tubular sleeve 48. The transparent tubular member 150 may be positioned at a fixed distance from the hub 120 of the vascular access sheath 100 regardless of the current length of the sterile sleeve 28 (e.g., regardless of the amount and/or degree of bunched location 52 along the tubular sleeve 48. The transparent tubular member 150 may have a fixed length while the tubular sleeve 48 may have an adjustable or variable length. The transparent tubular member 150 may have a column strength or rigidity greater than the column strength or rigidity of the tubular sleeve 48 such that the transparent tubular member 150 does not axially collapse when the tubular sleeve 48 axially collapses and bunches up when the connector 46 is moved toward the housing 14. The transparent tubular member 150may be formed of a transparent polymeric material permitting visualization of the elongate shaft 12 therein, whereas the tubular sleeve 48 may be a non-transparent material (e.g., a translucent material), obscuring visibility of the elongate shaft 12 therein.

The transparent tubular member 150 may be formed of one or more transparent materials, such as, but not limited to, polyamide, polyethylene (PE), polyethylene terephthalate (PET), polypropylene (PP), polytetrafluoroethylene (PTFE), fluorinated ethylene propylene (FEP), polyvinyl chloride (PVC), polyether block amides (PEBA), polycarbonate (PC), poly methyl methacrylate (PMMA), combinations thereof, and/or other suitable materials. In some instances, the transparent tubular member 150 may consist essentially of or consist entirely of a transparent polymeric material. The transparency of the transparent tubular member 150 may allow medical personnel to view the elongate shaft 12 (extending through the lumen of the transparent tubular member 150) through the transparent tubular member 150. The transparent tubular member 150 is more transparent than the tubular sleeve 48. For example, the transparent tubular member 150 may have a transmittance value of greater than 70%, whereas the tubular sleeve 48 may have a transmittance value of less than the transmittance value of the transparent tubular member 150, such as a transmittance value of less than 70%. Accordingly, the transparent tubular member 150 may have a transmittance value greater than the transmittance value of the tubular sleeve 48. In accordance with ASTM D-1003 (Standard Test Method for Haze and Luminous Transmittance of Transparent Plastics), the transparent tubular member 150 may have a Haze % value of less than 30%, whereas the tubular sleeve 48 may have a Haze % value of greater than the Haze % value of the transparent tubular member 150, such as a Haze % value of greater than 30%. Accordingly, the transparent tubular member 150 may have a Haze % value less than the Haze % value of the tubular sleeve 48.

The transparent tubular member 150 may facilitate visually viewing position markings 160 formed on the elongate shaft 12. Position markings 160, which may be formed at equidistant intervals along the length of the elongate shaft 12, may be used by medical personnel to determine the placement of the blood pump 50. For example, once the blood pump 50 has been positioned at a desired position within the heart of the patient (e.g., across the aortic valve and into the left ventricle) one of the position markings 160 may be noted, and then subsequently medical personnel may view the position markings 160 to confirm that the blood pump 50 is still in the desired position and has not be inadvertently moved out of position. For example, medical personnel may note a position of one of the position markings 160 relative to the hub 120 of the vascular access sheath 100, which position may be recorded, logged, etc. At subsequent times (e.g., hourly, daily, etc.), medical personnel may note the current position of the noted one of the position markings 160 relative to the hub 120 of the vascular access sheath 100 to confirm the position of the blood pump 50 or determine whether the blood pump 50 has been inadvertently moved.

The position markings 160 may be formed on the elongate shaft 12 by laser etching, ink, embossing, marker bands, etc. In some instances, the position markings 160 may include major markings 162 and minor markings 164 disposed at regular intervals along the length of the elongate shaft. For example, the major markings 162 may be positioned at every 5 centimeters along the length of the elongate shaft 12, while the minor markings may be positioned at every 1 centimeter between the major markings 162. The major markings 162 may include a numeric value representing a measured distance to the major marking 162 from a reference point of the percutaneous circulatory support device 10. For instance, the major markings 162 may include a numeric value (50, 60, 70, 80, 90, 100, etc.) representing the distance (in centimeters) that the major marking 162 is from the distal tip of the percutaneous circulatory support device 10.

In some instances, the transparent tubular member 150 may include an index marking 152 such that medical personnel can readily note the position of one of the position markings 160 (e.g., one of the major markings 162 and/or one of the minor markings 164) relative to the index marking 152. For example, medical personnel may note, document, record, and/or confirm which of the plurality of position markings 160 is aligned with the index marking 152 to determine a current position of the blood pump 50. The index marking 152 may be a line, an arrow, some other indicia, or the like, extending perpendicular to the longitudinal axis of the elongate shaft 12, for example. In other instances, the index marking 152 may be another distinguishing mark or indicia formed with or placed on the transparent tubular member 150.

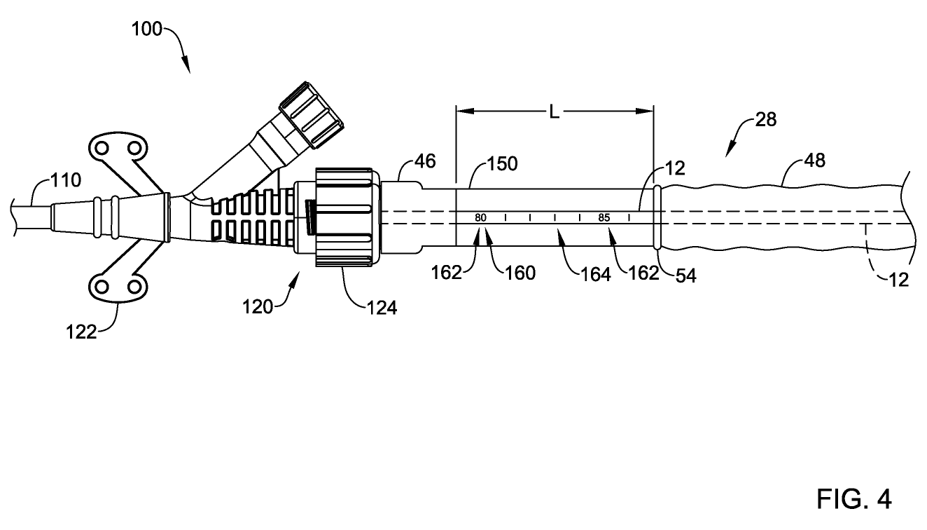

Turning to FIG. 4, the transparent tubular member 150 may have a longitudinal length L measured from a distal end of the transparent tubular member 150 to a proximal end of the transparent tubular member 150. In some instances, the length L may be 5 centimeters or more, 6 centimeters or more, 7 centimeters or more, 8 centimeters or more, 9 centimeters or more, or 10 centimeters or more. In some instances, it may be desirable that the length L of the transparent tubular member 150 be sufficient such that at least one of the major markings 162 (e.g., the markings with a numeric value) is visible through the tubular wall of the transparent tubular member 150 regardless of the axial position of the elongate shaft 12 within the transparent tubular member 150. Moreover, the length L of the transparent tubular member 150 may be the same as or greater than the distance between major markings 162 (e.g., the markings with a numeric value) formed on the elongate shaft 12 of the percutaneous circulatory support device 10. In embodiments in which the major markings 162 are spaced apart by a distance of 5 centimeters, the length L of the transparent tubular member 150 may be 5 centimeters or more, or 6 centimeters or more, for example.

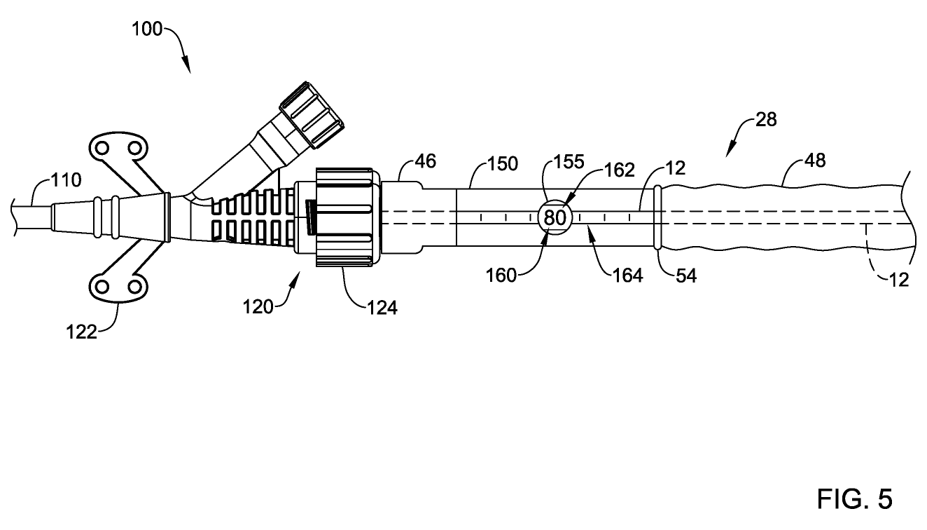

FIG. 5 illustrates features of another embodiment of the transparent tubular member 150. It is noted that features of this embodiment may be included in any other embodiment described herein, if desired. Likewise, any features of another embodiment described herein, may be included in this embodiment, if desired. As shown in the embodiment of FIG. 5, the transparent tubular member 150 may include a magnification window 155 providing a magnified view of the position markings 160 formed along the elongate shaft 12. In some instances, the magnification window 155 may have a magnification power of 1.5X or more, 2.0X or more, 2.5X or more, or 3X or more, such that the position markings 160 are enlarged in the magnification window 155 to a size greater than the actual size of the position markings 160 to facilitate viewing of the position marking(s) 160 visible through the magnification window 155. FIG. 5 illustrates a major marking 162 (e.g., a marking with a numeric value) visible through the magnification window 155 such that the size of major marking 162 as viewed through the magnification window 155 is greater than the actual size of the major markings 162 formed along the elongate shaft 12.

In some instances, the magnification window 155 may be a magnified lens inset into the transparent tubular member 150 having a magnification power of 1.5X or more. In some instances, the magnification window 155 may be centered between the proximal and distal ends of the transparent tubular member 150, or the magnification window 155 may be positioned at a different position along the length of the transparent tubular member 150. The magnification window 155 may be located at a fixed distance from the hub 120 of the vascular access sheath 100. In some instances, the magnification window 155 may be the only transparent portion of the transparent tubular member 150. However, in other instances, the remainder of the transparent tubular member 150 (i.e., the portion of the transparent tubular member 150 surrounding the magnification window 155 may also be formed of a transparent material.

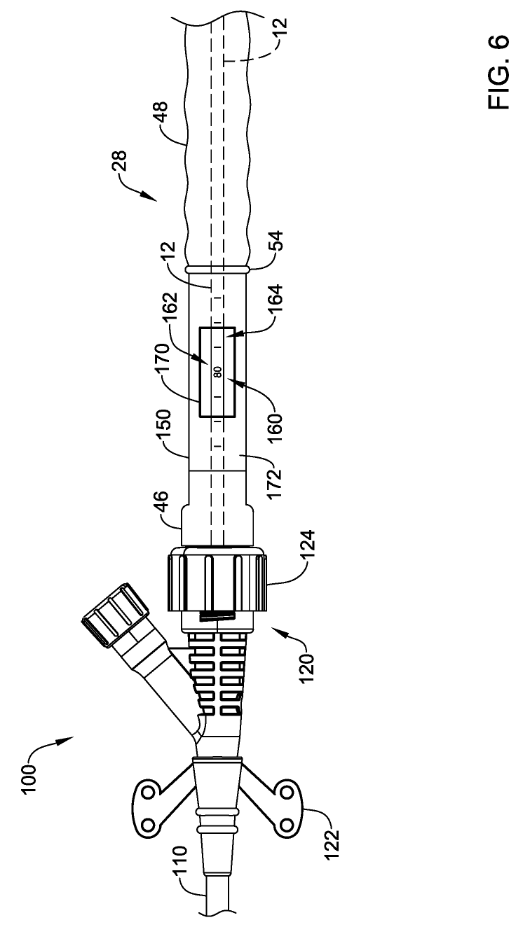

FIG. 6 illustrates features of another embodiment of the transparent tubular member 150. It is noted that features of this embodiment may be included in any other embodiment described herein, if desired. Likewise, any features of another embodiment described herein, may be included in this embodiment, if desired. As shown in the embodiment of FIG. 6, the transparent tubular member 150 may include a transparent viewing window 170 providing a clear view of the position markings 160 formed along the elongate shaft 12. In some instances, the transparent viewing window 170 may be a lens inset into the tubular member 172 forming transparent tubular member 150. In some instances, the transparent viewing window 170 may be centered between the proximal and distal ends of the transparent tubular member 150, or the transparent viewing window 170 may be positioned at a different position along the length of the transparent tubular member 150. The transparent viewing window 170 may be located at a fixed distance from the hub 120 of the vascular access sheath 100. In some instances, the transparent viewing window 170 may be the only transparent portion of the transparent tubular member 150. For instance, the transparent viewing window 170 may be surrounded by a non-transparent tube 172 forming the remainder of the transparent tubular member 150. However, in other instances, the remainder of the transparent tubular member 150 (i.e., the portion of the transparent tubular member 150 surrounding the transparent viewing window 170) may also be formed of a transparent material, or may be formed of a translucent material. In some instances, the transparent viewing window 170 may be more transparent than the remainder of the transparent tubular member 150 surrounding the transparent viewing window 170. In other words, the transparent viewing window 170 may have a transmittance value greater than the transmittance value of the remainder of the transparent tubular member 150 surrounding the transparent viewing window 170 and/or the transparent viewing window 170 may have a Haze % value less than the Haze % value of the remainder of the transparent tubular member 150 surrounding the transparent viewing window 170.

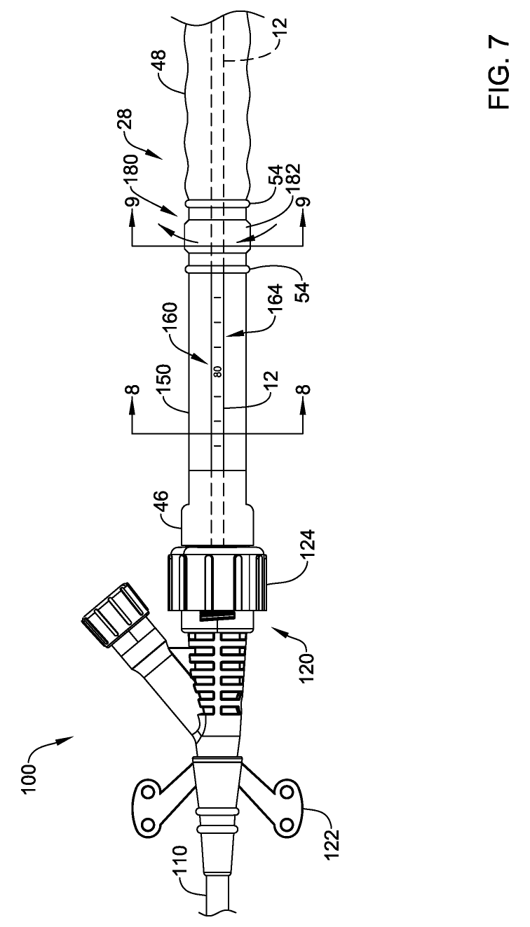

FIG. 7 illustrates features of another embodiment of the sterile sleeve 28 including the transparent tubular member 150. It is noted that features of this embodiment may be included in any other embodiment described herein, if desired. Likewise, any features of another embodiment described herein, may be included in this embodiment, if desired.

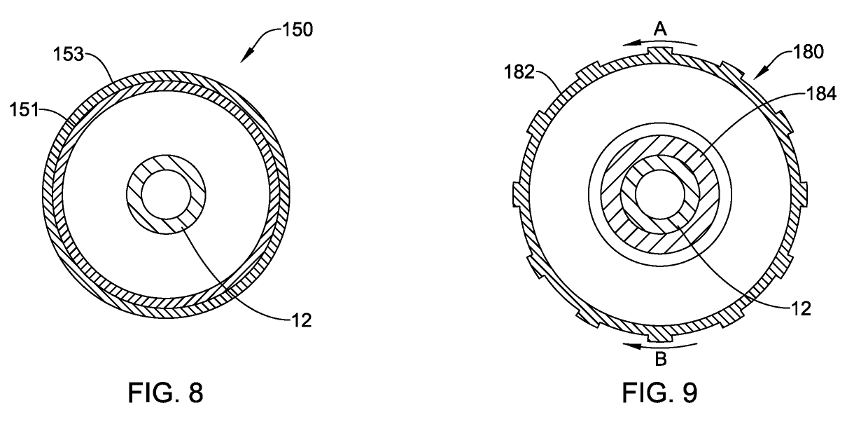

As shown in the cross-section of FIG. 8, which is a cross-sectional view taken along line 8-8 of FIG. 7, the transparent tubular member 150 may be a multi-layer tubular member including a first, inner layer 151 defining the lumen through the transparent tubular member 150 and a second, outer layer 153 defining an outer surface of the transparent tubular member 150. The multilayer tubular member may also include additional layers, such as one or more intermediate layers between the inner layer 151 and the outer layer 153, if desired. In some instances, the inner layer 151 may be formed of a first transparent material and the outer layer 153 may be formed of a second transparent material different from the first transparent material. Each of the first and second transparent material may be any desired transparent material, including, but not limited to, the transparent polymeric materials disclosed herein. In some instances, the inner layer 151 may be formed of a transparent polytetrafluoroethylene (PTFE) while the outer layer 153 may be formed of a transparent polyether block amide (PEBA).

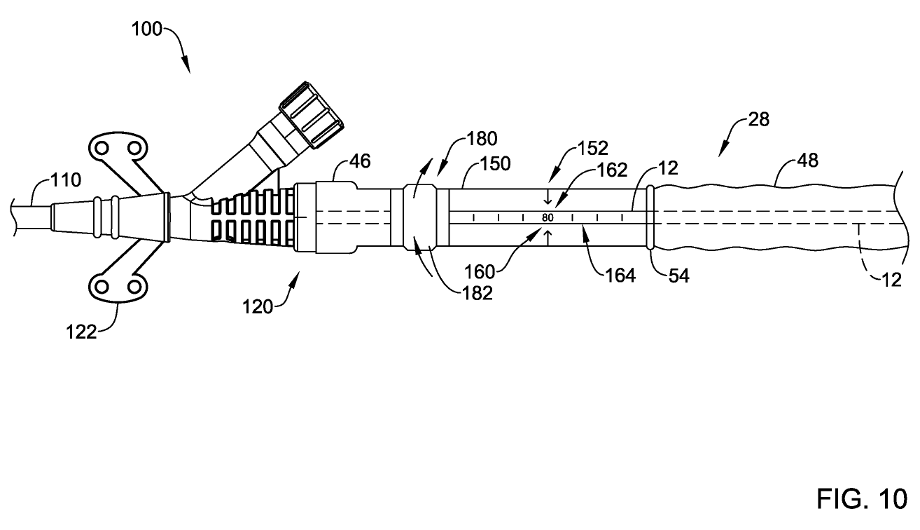

Furthermore, as shown in the embodiment of FIG. 7, the sterile sleeve 28 may include an adjustable tightening port 180, provided with or attachable thereto, that provides for the selectable fixation of the percutaneous circulatory support device 10 (e.g., the elongate shaft 12) with respect to the sterile sleeve 28, and thus with respect to the hub 120. In some instances, the tightening port 180 may be provided in addition to or in place of the tightening port 124 in the hub 120 of the vascular access sheath 100. The tightening port 180 may be provided at any location along the sterile sleeve 28. For example, the tightening port 180 may be positioned between the transparent tubular member 150 and the tubular sleeve 48. In other embodiments, such as shown in FIG. 10, the tightening port 180 may be positioned between the transparent tubular member 150 and the hub 120 of the vascular access sheath 100. In some instances, a connector 54 may be used to couple the tightening port 180 to the transparent tubular member 150 and/or a connector 54 may be used to couple the tightening port 180 to the tubular sleeve 48. The tightening port 180 of the sterile sleeve 28 may be any suitable type of tightening port, which may be attachable to and/or detachable from the hub 120 of the vascular access sheath 100. For example, the tightening port 180 may tighten around and/or release from the elongate shaft 12 in response to rotating a rotational actuator 182 (e.g., a rotatable knob) of the tightening port 180. As shown in the cross-section of FIG. 9, which is a cross-sectional view taken along line 9-9 of FIG. 7, the elongate shaft 12 may extend through a compressible seal 184 of the adjustable tightening port 180. Rotation of the rotatable actuator 182 in a first rotational direction (shown by Arrow A in FIG. 9) may radially compress the compressible seal 184 against an outer surface of the elongate shaft 12 to secure the position of the elongate shaft 12 relative to the adjustable tightening port 180, and thus relative to the transparent tubular member 150 of the sterile sleeve 28. Rotation of the rotatable actuator 182 in an opposite, second rotational direction (shown by Arrow B in FIG. 9) may release the compressive force applied to the elongate shaft 12 from the compressible seal 184, enlarging the lumen through the compressible seal 184 to permit the elongate shaft 12 to be moved axially through the adjustable tightening port 180, and thus moved axially through the transparent tubular member 150 of the sterile sleeve 28 to adjust a position of the blood pump 50. It is noted that the tightening port 180 may adjust about the elongate shaft 12 in additional or alternative manners.

Another arrangement of the tightening port 180 with the sterile sleeve 28 is shown in FIG. 10, which illustrates the tightening port 180 positioned distal of the transparent tubular member 150, and thus between the transparent tubular member 150 and the hub 120 of the vascular access sheath 100. In such instances, the hub 120 of the access sheath 100 may not include its own tightening port, but rather may include a passive hemostasis valve, for example. The sterile sleeve 28 may include a distal connector 46 coupled to the hub 120 of the vascular access sheath 100, with the tightening port 180 located proximally of the connecter 46. A distal end of the transparent tubular member 150 may be secured to the tightening port 180 and a proximal end of the transparent tubular member 150 may be secured to the tubular sleeve 48, such as via a connector 54. The tightening port 180 of the sterile sleeve 28 may be any suitable type of tightening port, which may be attachable to and/or detachable from the hub 120 of the vascular access sheath 100. For example, the tightening port 180 may tighten around and/or release from the elongate shaft 12 in response to rotating a rotational actuator 182 (e.g., a rotatable knob) of the tightening port 180, as described above. It is noted that features of this embodiment may be included in any other embodiment described herein, if desired. Likewise, any features of another embodiment described herein, may be included in this embodiment, if desired.

During a medical procedure, the blood pump 50 may be advanced distally through the vascular access sheath 100 (or a different vascular access sheath) and to a desired position within the patient (e.g., across the aortic valve in the heart of the patient and into the left ventricle of the heart of the patient), or the blood pump 50 may be otherwise located at a desired position within the patient with the elongate shaft 12 of the percutaneous circulatory support device 10 extending through the vascular access sheath 100. It is noted that in some instances the blood pump 50 may be initially advanced into the vasculature of the patient using a different vascular access sheath and thereafter exchanged for the vascular access sheath 100 with the elongate shaft 12 of the percutaneous circulatory support device 10 extending through the vascular access sheath 100. With the elongate shaft 12 extending through the vascular access sheath 100 and the blood pump 50 positioned at a desired position within the subject, the sterile sleeve 28 may remain proximal of the vascular access sheath 100, including the hub 120 of the vascular access sheath 100. The sterile sleeve 28 may extend over the elongate shaft 12 to create a sterile boundary over the elongate shaft 12 from the hub 120 to the housing 14. In some cases, the connector 46 of the sterile sleeve 28 may couple with the hub 120, as discussed above.

The vascular access sheath 100 may be fixed relative to the access site of the subject, by suturing the hub 120 to the skin of the patient proximate the access site with one or more sutures sutured between the suture pads 122 and the skin of the patient, or otherwise coupled to a skin surface of the subject.

Medical personnel may fix the axial position of the percutaneous circulatory support device 10 relative to the vascular access sheath 100 via the adjustable tightening port 124 (or the tightening port 180 if provided with the sterile sleeve 28) once the blood pump 50 has been positioned at a desired position (e.g., across the aortic valve of the heart of the patient). For example, the tightening port 124 (or the tightening port 180 if provided with the sterile sleeve 28) may tighten around the elongate shaft 12 in response to rotating a rotational actuator of the tightening port 124/180, to thereby secure the percutaneous circulatory support device 10 relative to the vascular access sheath 100. Medical personnel may note, document, record, or otherwise confirm the position of the elongate shaft 12 relative to the transparent tubular member 150 of the sterile sleeve 28 when the blood pump is properly positioned and operating as desired. For example, the elongate shaft 12 may include a plurality of position markings which may be used to denote the axial position of the elongate shaft 12 relative to the hub 120 of the vascular access sheath 100, and thus the transparent tubular member 150 of the sterile sleeve 28 coupled thereto. An index marking, when the transparent tubular member is provided therewith, may be used as a reference point of an axial position of the elongate shaft 12 within the transparent tubular member 150.

At one or more temporal occasions during usage of the percutaneous circulatory support device 10, medical personnel may visually inspect the axial position of the elongate shaft 12 of the percutaneous circulatory support device 10 relative to the vascular access sheath 100 to confirm the proper position of the blood pump 50, or determine if the blood pump 50 has been inadvertently moved during use. For example, once the blood pump 50 has been positioned at a desired position within the heart of the patient (e.g., across the aortic valve and into the left ventricle) one of the position markings 160 may be noted, and then subsequently medical personnel may view the position markings 160 to confirm that the blood pump 50 is still in the desired position and has not be inadvertently moved out of position. For example, medical personnel may note a position of one of the position markings 160 relative to the hub 120 of the vascular access sheath 100, which position may be recorded, logged, etc. At subsequent times (e.g., hourly, daily, etc.), medical personnel may note the current position of the noted one of the position markings 160 relative to the hub 120 of the vascular access sheath 100 to confirm the position of the blood pump 50 or determine whether the blood pump 50 has been inadvertently moved. In the event that the blood pump 50 has moved (i.e., the noted position of the position markings 160 on the elongate shaft 12 have changed relative to the vascular access sheath 100 and/or the transparent tubular member 150 of the sterile sleeve 28), medical personnel can adjust the axial position of the elongate shaft 12 back to the initial noted position of the position marking 160 to thereby reposition the blood pump 50 back to its desired position within the patient.

The schematic methods and techniques discussed herein may be used together, unless expressly indicated otherwise. Further, the order of implementation of the steps discussed herein may be in other suitable orders, unless expressly indicated otherwise, and/or the methods and techniques may include one or more intervening steps discussed herein or otherwise.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A percutaneous circulatory support system, the system comprising:
a blood pump configured to pump blood from a ventricle of a heart of a patient to vasculature of the patient;
a housing;
an elongate shaft coupled with the blood pump and extending proximally from the blood pump to the housing; and
a sterile sleeve configured to extend over the elongate shaft distal of the housing, the sterile sleeve including:
a tubular sleeve having an adjustable length; and
a transparent tubular member extending distally of the tubular sleeve;
wherein the elongate shaft is visible through the transparent tubular member.

2. The percutaneous circulatory support system of claim 1, wherein the transparent tubular member has a fixed length.

3. The percutaneous circulatory support system of claim 2, wherein the elongate shaft includes a plurality of position markings, the plurality of position markings including major markings having numeric values, and
wherein at least one of the major markings having numeric values is visible through the transparent tubular member regardless of the axial position of the elongate shaft therein.

4. The percutaneous circulatory support system of claim 3, wherein the major markings having numeric values are spaced 5 centimeters apart, and wherein the fixed length of the transparent tubular member is 5 centimeters or more.

5. The percutaneous circulatory support system of claim 1, wherein the transparent tubular member includes a magnification window.

6. The percutaneous circulatory support system of claim 5, wherein the magnification window has a magnification power of 1.5X or more.

7. The percutaneous circulatory support system of claim 1, wherein the tubular sleeve includes a plurality of bunched locations.

8. The percutaneous circulatory support system of claim 1, wherein the transparent tubular member includes an index marking configured to be used as a reference point of an axial position of the elongate shaft within the transparent tubular member.

9. The percutaneous circulatory support system of claim 8, wherein the elongate shaft includes a plurality of position markings, wherein the position of one of the plurality of position markings relative to the index marking is visible through the transparent tubular member.

10. The percutaneous circulatory support system of claim 1, wherein the transparent tubular member includes a transparent viewing window surrounded by a non-transparent tube.

11. The percutaneous circulatory support system of claim 1, wherein the transparent tubular member is formed of a transparent polymeric material permitting visualization of the tubular shaft therein and the tubular sleeve is formed of a translucent material obscuring visibility of the tubular shaft therein.

12. The percutaneous circulatory support system of claim 11, wherein the transparent tubular member has a Haze % value of less than 30% in accordance with ASTM D-1003, and the tubular sleeve has a Haze % value of greater than the Haze % value of the transparent tubular member.

13. The percutaneous circulatory support system of claim 1, further comprising:
a vascular access sheath including a hub and an elongate tubular shaft extending distally from the hub;
wherein the sterile sleeve is coupled to the hub of the vascular access sheath via a connector.

14. The percutaneous circulatory support system of claim 13, wherein the transparent tubular member is located at a fixed distance proximal of the hub.

15. The percutaneous circulatory support system of claim 13, wherein the transparent tubular member includes an index marking located at a fixed distance proximal of the hub.

16. A percutaneous circulatory support system, the system comprising:
a percutaneous circulatory support device including:
a blood pump configured to pump blood from a ventricle of a heart of a patient to vasculature of the patient;
a housing; and
an elongate shaft coupled with the blood pump and extending proximally from the blood pump to the housing, the elongate shaft including a plurality of position markings formed along a length of the elongate shaft; and
a sterile sleeve configured to extend over the elongate shaft distal of the housing, the sterile sleeve including:
a tubular sleeve having a plurality of bunched locations providing the tubular sleeve with an adjustable length; and
a transparent tubular member extending distally of the tubular sleeve, the transparent tubular member having a fixed length;
wherein one or more of the position markings is visible through the transparent tubular member to denote a position of the percutaneous circulatory support device.

17. The percutaneous circulatory support system of claim 16, wherein the plurality of position markings including major markings having numeric values, and
wherein at least one of the major markings having numeric values is visible through the transparent tubular member regardless of the axial position of the elongate shaft therein.

18. The percutaneous circulatory support system of claim 16, wherein the transparent tubular member includes an index marking configured to be used as a reference point of an axial position of the elongate shaft within the transparent tubular member, and
wherein the position of one of the plurality of position markings relative to the index marking is visible through the transparent tubular member.

19. A percutaneous circulatory support system, the system comprising:
a percutaneous circulatory support device including:
a blood pump configured to pump blood from a ventricle of a heart of a patient to vasculature of the patient;
a housing; and
an elongate shaft coupled with the blood pump and extending proximally from the blood pump to the housing, the elongate shaft including a plurality of position markings formed along a length of the elongate shaft; and
a vascular access sheath including a hub and an elongate tubular shaft extending distally of the hub;
a sterile sleeve configured to extend over the elongate shaft distal of the housing and be coupled to the hub of the vascular access sheath with the elongate shaft of the percutaneous circulatory support device extending through the vascular access sheath, the sterile sleeve including:
a tubular sleeve formed of a non-transparent material; and
a transparent tubular member extending distally of the tubular sleeve, the transparent tubular member comprising a transparent material;
wherein one or more of the position markings is visible through the transparent tubular member to denote a position of the blood pump of the percutaneous circulatory support device relative to the hub of the vascular access sheath.

20. The percutaneous circulatory support system of claim 19, wherein the transparent tubular member includes an index marking configured to be used as a reference point of an axial position of the elongate shaft within the transparent tubular member.
